(19) United States
(12) Patent Application Publication
(10) Pub. No.: US 2022/0008884 A1
(43) Pub. Date: Jan. 13, 2022

(54) CATALYST BED COMPRISING SILVER CATALYST BODIES AND PROCESS FOR THE OXIDATIVE DEHYDROGENATION OF OLEFINICALLY UNSATURATED ALCOHOLS

(71) Applicant: BASF SE, Ludwgshfen am Rhein (DE)

(72) Inventors: Nicolas DUYCKAERTS, Ludwgshfen am Rhein (DE); Martin KAMASZ, Ludwgshfen am Rhein (DE); Christian WALSDORFF, Ludwgshfen am Rhein (DE)

(21) Appl. No.: 17/293,647

(22) PCT Filed: Nov. 12, 2019
(86) PCT No.: PCT/EP2019/081000

§ 371 Date: May 13, 2021

(30) Foreign Application Priority Data
Nov. 13, 2018 (EP) 18205985.7

(51) Int. Cl. B01J 8/06; C07C 45/38; B01J 35/02; B01J 23/50; B01J 35/10; B01J 35/08

(52) U.S. Cl. CPC B01J 8/067; C07C 45/38; B01J 35/023; B01J 2208/027; B01J 35/1019; B01J 35/08; B01J 2208/024; B01J 23/50

(57) ABSTRACT

The present invention relates to a catalyst bed comprising silver catalyst bodies and a reactor comprising such a catalyst bed. Further, the invention relates to the use of the catalyst bed and the reactor for gas phase reactions, in particular for the oxidative dehydrogenation of organic compounds under exothermic conditions. In a preferred embodiment, the present invention relates to the preparation of olefinically unsaturated carbonyl compounds from olefinically unsaturated alcohols by oxidative dehydrogenation utilizing a catalyst bed comprising metallic silver catalyst bodies.

### FIELD OF THE INVENTION

The present invention relates to a catalyst bed comprising silver catalyst bodies and a reactor comprising such a catalyst bed. Further, the invention relates to the use of the catalyst bed and the reactor for gas phase reactions, in particular for the oxidative dehydrogenation of organic compounds under exothermic conditions. In a preferred embodiment, the present invention relates to the preparation of olefinically unsaturated carbonyl compounds from olefinically unsaturated alcohols by oxidative dehydrogenation utilizing a catalyst bed comprising metallic silver catalyst bodies.

### BACKGROUND OF THE INVENTION

Catalytic gas phase reactions are an important class of well established chemical processes that lead to a broad variety of different intermediates and products of value. A chemical reaction that is often performed as a catalytic gas phase reaction is the oxidative dehydrogenation. Typical catalysts used in catalytic gas phase reaction are metal catalysts, like silver. The preparation of alpha-beta-unsaturated carbonyl compounds by oxidative dehydrogenation over suitable catalysts is known to those skilled in the art and has been widely described in the literature.

Catalyst performance is characterized e.g. by conversion, selectivity, activity, longevity of catalyst activity, and mechanical stability. Moreover, the performance in the reactor tubes is characterized by the packing density of the catalyst in the volume of the tubes and pressure drop across the catalyst bed. To be considered satisfactory, a catalyst must not only have a sufficient activity and the catalytic system provide an acceptable selectivity, but the catalyst must also demonstrate an acceptable lifetime or stability. When a catalyst is spent, typically the reactor must be shut down and partially dismantled to remove the spent catalyst. This results in losses in time and productivity. In addition, the catalyst must be replaced and in case of metal catalysts, like silver, the silver recovered or, where possible, regenerated. Even small improvements in selectivity and/or activity and in the maintenance of selectivity and/or activity over longer time yield huge dividends in terms of process efficiency.

The oxidative dehydrogenation of olefinically unsaturated alcohols to olefinically unsaturated aldehydes is highly exothermic. The control of the reaction is difficult because the reaction rate depends strongly on the reaction temperature, and the reactants as well as the products that are unstable under the reaction conditions. This can lead to coke formation, which accumulates over time and leads to the necessity for a periodical regeneration of the catalyst by combustion of the coke deposits using an oxygen containing gas stream, to ensure safe operation. It has also to be considered that gas phase reactions of a combustible organic compound and oxygen generally bear the risk of reaching an explosive area.

A general problem that is associated with the use of catalyst beds in a multitubular reactor is to ensure a narrow pressure drop distribution along all the individual tubes of the reactor. This can be generally be improved by using catalyst particles with a narrow particle size distribution. If in the course of the oxidative dehydrogenation also coke particles are formed, the inhomogeneity of the gas flow may become even larger and may reach a point where a part of the tubes are fully clogged and are lost for the aldehyde production process. Furthermore, fully clogged reactor tubes are difficult to regenerate (e.g. by burning of the coke in the presence of air) as no gas can flow through. This, in turn, can lead to the formation of hotspots in coked tubes, where only a minor amount of flow can pass through.

U.S. Pat. No. 2,042,220 discloses oxidizing 3-methyl-3-butene-1-ol (isoprenol) with an excess of oxygen in the presence of metal catalysts, for example copper and silver catalysts, to form 3-methyl-3-buten-1-al (isoprenal). The catalysts can be alloys, metal compounds or elemental metal. Activated catalysts are preferred; activating options include surface amalgamation of the metal and subsequent heating of the metal surface. In the examples, copper and silver catalysts are prepared by reducing copper oxide particles under hydrogen or by amalgamation and heating of silver wire networks. According to DE 20 41 976, the process of U.S. Pat. No. 2,042,220 leads to appreciable amounts of undesirable by-products.

U.S. Pat. No. 4,165,342 discloses oxidizing 3-methyl-3-butene-1-ol (isoprenol) with an excess of oxygen in the presence of metal catalysts, for example copper and silver catalysts, to form 3-methyl-3-buten-1-al (isoprenal). The catalysts are used in the form of metallic copper or silver crystals with various size distributions. Silver crystals are characterized by a low packing density.

EP 0 263 385 relates to a process for the oxidative dehydrogenation of 3-methyl-3-butene-1-ol to 3-methyl-3-butene-3-al in the gas-phase in the presence of a silver catalyst. The silver catalyst is obtained by flame spray synthesis, where silver powder is melted and brought on steatite spheres having a diameter of 0.16 to 0.20 cm, leading to a final catalyst with a silver content of about 4 wt.-% of silver.

EP 0 881 206 relates to a continuous industrial production of unsaturated aliphatic aldehydes by oxidative dehydrogenation of the corresponding alcohols with an oxygen-comprising gas over a supported catalyst consisting of copper, silver and/or gold on an inert support in a tube bundle reactor, rapid cooling of the reaction gases and removal of the aldehydes from the resulting condensate.

WO 2008/037693 relates to the preparation of 3-methyl-2-butenal by oxidative dehydrogenation of 3-methyl-2-butenol, in a sand bath-heated short tube reactor, using a silver supported catalyst with 6 wt.-% of silver.

WO 2009/115492 relates to the use of a supported catalyst comprising noble metals for the oxidative dehydrogenation of isoprenol. The supported catalyst contains e.g. silver on steatite spheres, has a silver content of 6 wt.-% and a particle diameter of 0.18 to 0.22 cm. It is synthesized by a flame spray or by applying a complex solution of ethylenediamine and silver oxalate with subsequent drying in an air stream.

EP 2 448 669 relates to a supported catalyst comprising noble metals, e. g. silver. The silver metal-containing catalysts are obtained by applying colloidal silver to steatite spheres.

WO 2012/146436 relates to a catalyst of silver coated steatite spheres for the oxidative dehydrogenation of 3-methyl-3-butene-1-ol to 3-methyl-3-butene-3-al.

CN 103769162 relates to a composite metal catalyst used for the oxidation of unsaturated alcohols and a method for the preparation thereof. The catalyst comprises: 0.001 to 0.3 wt.-% of an alkali metal, 0.001 to 1 wt.-% of an alkaline earth metal, 0.001 to 1 wt.-% of scandium, 0.05 to 1 wt.-% of cerium oxide and zirconia sol, 0.3 to 10 wt.-% of copper, 1 to 30 wt.-% of silver, and 60 to 95 wt.-% of a carrier.

The production of coated silver catalysts, e.g. on steatite or other inert support materials is tedious, complex and expensive. A significant separation effort is required to recycle and recover silver from the spent catalysts. This also leads to the generation of large amounts of support material as effluent on an industrial scale. Also, depending on the coating process used, the mechanical instability of the coated silver can lead to silver losses by attrition.

CN 108404944 relates to a method for the preparation of a vanadium silver molybdenum phosphate catalyst and using this catalyst for the preparation of a prenal.

EP 0 055 354 describes the oxidative dehydrogenation of 3-alkylbuten-1-ols over catalysts consisting of layers of silver and/or copper crystals in the presence of molecular oxygen. The process makes use of an adiabatic reactor for the oxidative dehydrogenation of the corresponding alcohol in the presence of a structured catalyst bed with four layers of silver crystallites, each having a different particle size distribution. The first three layers account for 95 wt.-% of the catalyst and have a total particle size distribution of 0.02 to 0.1 cm. The disadvantage of this process is that good selectivities can only be achieved if defined catalyst particle sizes or a defined particle size distribution are used in a specific layer construction. This generally increases the cost of catalyst, which is filled into the reactor. A further disadvantage of a layered structure is an inhomogenious distribution of the residence time of the gas feed in contact with the catalyst packing. In addition, the high reaction temperatures employed give rise to sintering of the metal crystals, which leads to pressure buildup and shorter onstream times.

EP 0 244 632 relates to a tube bundle reactor for carrying out organic reactions, e. g. a process for preparing aliphatic, aromatic or araliphatic ketones and aldehydes in the gas phase. The thickness of the catalyst bed ranges from 10 to 150 mm. The catalyst particles are dumped for example onto a silver or stainless steel mesh with the reactor in the upright position. The catalyst particles have a particle size of from 0.1 to 5 mm. This document does not disclose to employ catalyst particles having a packing density in the range of 3.0 g/cm³ to 10.0 g/cm³ and essentially spherical shape.

Enhong Cao et al. describe in Chemical Engineering Science 59 (2004) 4803-4308 the oxidative dehydrogenation of 3-methyl-2-buten-1-ol in silicon-glass silver-coated micro-reactors. The industrial applicability of silver-coated microreactors either of massive silver or coated with silver is economically not viable. Depending on the coating process, silver abrasion might also become problematic with all its consequences.

U.S. Pat. No. 4,390,730 describes the production of formaldehyde by oxidative dehydrogenation of methanol in the presence of a lead-silver catalyst. Indeed U.S. Pat. No. 4,390,730 discloses the methanol oxidation using an unpromoted silver catalyst (mesh size silver crystals). Nevertheless, the experimental results show that the lead-silver catalysts provide higher efficiencies for methanol conversion to formaldehyde.

WO 01/30492 describes a crystalline silver catalyst for the preparation of formaldehyde via methanol conversion. The silver catalyst has a packing density of less than 2.5 g/L.

WO 01/30492 discloses as comparative examples silver catalysts having a packing density of more than 2.5 g/L. It has been shown that a lower silver packing density (less than 2.5 g/L) leads to a better catalytic activity.

U.S. Pat. No. 4,450,301 describes a process for oxidizing methanol to formaldehyde in the presence of two sequential silver-based catalysts.

US 2017/0217868 describes a silver catalyst for the conversion of methanol to formaldehyde. The silver can be used in bulk form (gauzes screens, powders or shots).

However, the last five documents relate only to the prepartion of formaldehyde via conversation of methanol. These documents are silent about the preparation of olefinically unsaturated carbonyl compounds.

US 2003/159799 describes the oxidation of 3-methyl-3-buten-1-ol to 3-methyl-2-butenal in the presence of a silver catalyst. This silver catalyst is prepared by coating a woven tape of heat-resistant stainless steel with silver in an electron beam vapor deposition unit. In other words a silver coated woven metal tape is obtained. A full-metallic silver catalyst body is not mentioned.

U.S. Pat. No. 5,149,884 describes a tube bundle reactor for carrying out catalytic organic reaction, e.g. prepartion of ketones and aldehydes in the gase phase, wherein the tubes have certain dimensions. The catalyst particles are dumped onto a silver or stainless steel mesh with the reactor in the upright position. The catalysts are silver particles having a size ranging from 0.1 to 5 mm. The document contains no information about the nature of the catalyts or packing density, etc.

WO 2012/146528 describes describes a process for the preparation of C₁-C₁₀-aldehydes by oxidative dehydrogenation of the corresponding alcohols in the presence of a shaped catalyst body which is obtainable by three-dimensional deformation and/or arrangement in the space of silver-containing fibres and/or filaments. The mean diameter or the mean diagonal length of a substantially rectangular or square cross-section of these silver-containing fibres and/or filaments is in the range from 30 to 200 μm. The density of the shaped fibers is in the range of 2 to 4 g/cm³. The three-dimensional deformation and/or arrangement of the silver-containing fibres or threads in space can take place in an unordered or ordered manner. The disorderly deformation and/or arrangement of the silver-containing fibres leads to balls. An ordered deformation and/or arrangement of the silver-containing fibres is obtained by knitting or weaving.

WO 2018/153736 describes a silver containing catalyst for the preparation of aldehyds and ketones, in particular the preparation of formaldehyde, by oxidative dehydrogenation of methanol. The catalyst is a two layer system. The first catalyst layer consists of silver-containing material in form of bundels, nets or meshes having a weight per unit of 0.3 to 10 kg/m² and a wire diameter of 30 to 200 μm. The second layer consists of silver-containing material in form of granulates having a particle size of 0.5 to 5 mm. The three-dimensional deformation and/or arrangement of the silver-containing fibres or threads in space can take place in an unordered or ordered manner. The granulate is a granular material consisting of small, usually irregularly shaped particles, e.g. silver crystals.

It is an object of the present invention to provide a catalyst bed having improved properties that is suitable for the use in catalytic gas phase reactions and in particular for the oxidative dehydrogenation of unsaturated alcohols to unsaturated carbonyl compounds. With the provision of said catalyst bed at least some of the afore-mentioned disadvantages shall be overcome. In particular the catalyst bed and the process making use thereof should have at least one of the following advantages:
The catalyst bed should be suitable for catalytic gas phase reactions having a high selectivity with regard to the desired product of value. In particular in the oxidative dehydrogenation of isoprenol a high selectivity with regard to prenal and isoprenal shall be obtained.
In tube bundle reactors an equal flow over the different reactor tubes shall be obtained. The formation of unwanted hot spots and or the clogging of reactor tubes shall be avoided.
A reactor containing the catalyst bed shall have advantageous heat transport properties allowing in particular an effective transfer of the heat of reaction to the surrounding heat transfer medium.
The production costs and/or the costs for regenerating the spent catalyst shall be low.
The catalyst shall be mechanically stable and in particular must not show abrasive mass loss.

A general problem that is associated with the use of metal catalyst beds found in the prior art, such as silver metal crystallites, in a multitubular reactor results from the usually broad particle size distribution of the silver metal crystallites combined with the low packing density obtained. This broad particle size distribution leads to an inhomogeneity of the gas flow through the different tubes of the reactor. This limits the practical application of silver crystallites as catalyst for these types of reactions.

It is further an object of the present invention to provide a process for the preparation of an olefinically unsaturated carbonyl compound which is efficient and selective towards the desired reaction product.

It has now been found that, surprisingly, a catalyst bed composed of silver catalyst bodies with very good performance properties can be obtained by the use of full-metallic silver catalyst bodies having a high packing density of the catalyst bodies in the range of 3.0 g/cm³ to 10.0 g/cm³.

It has further been found that, surprisingly, a process for the preparation of an olefinically unsaturated carbonyl compound in a tubular reactor in the presence of a catalyst bed comprising full-metallic silver catalyst bodies having a packing density in the range of 3.0 g/cm³ to 10.0 g/cm³ is more efficient and selective towards the desired reaction product compared to state of the art processes and catalyst beds.

### SUMMARY OF THE INVENTION

The invention provides a process for the preparation of an olefinically unsaturated carbonyl compound in a tubular reactor comprising a plurality of reactor tubes, comprising reacting an olefinically unsaturated alcohol with oxygen in the presence of a catalyst bed, comprising full-metallic silver catalyst bodies, wherein the catalyst bed has a packing density of the full-metallic silver catalyst bodies in the range of 3.0 g/cm³ to 10.0 g/cm³, preferably in the range of 5.5 g/cm³ to 10.0 g/cm³.

The invention further provides a catalyst bed comprising full-metallic silver catalyst bodies, wherein the catalyst bed has a packing density of the full-metallic silver catalyst bodies in the range of 5.5 g/cm³ to 10.0 g/cm³, preferably 6.0 g/cm³ to 10 g/cm³.

Preferably, the catalyst bed is in the form of a single layer characterized by an essentialy homogenious distribution of the packing density of the full-metallic silver catalyst bodies.

Preferably, the catalyst bed is in the form of a single layer characterized by an essentialy homogeneous distribution of the particle size of the full-metallic silver catalyst bodies.

Preferably, the catalyst bed does not contain two or more different layers, wherein the distribution of the packing density of each layer is different from those of the other layers.

Preferably, the catalyst bed does not contain two or more different layers, wherein the distribution of the particle size of each layer is different from those of the other layers.

Preferably, the catalyst bed according to the invention is located in a tube reactor, more preferably in the reactor tubes of a tube bundle reactor.

The invention further provides a reactor, comprising a plurality of reactor tubes containing a catalyst bed as defined above and in the following.

The invention further provides the use of a catalyst bed as defined above and in the following for the preparation of olefinically unsaturated carbonyl compounds from olefinically unsaturated alcohols by oxidative dehydrogenation.

### DETAILED DESCRIPTION OF THE INVENTION

The catalyst bed according to the invention, comprising a plurality of full-metallic silver catalyst bodies, is located in a chemical reaction vessel (reactor) suitable for continuous gas phase reactions. Generally, the reactor has at least two openings, at least one for allowing chemical compounds to pass into and at least one for allowing the products to be discharged out of the reactor. Further, the reactor is suitable for carrying out a chemical reaction, comprising the step of contacting one or more starting chemical compounds with the catalyst bed according to the invention to form at least one product chemical compound. The chemical reaction may comprise any of a large number of known chemical transformations, in particular catalytic gas-phase reactions, including e.g. (partial) oxidation, hydrogenation, dehydrogenation, oxidative dehydrogenation, etc. The nature of the reactor is usually not critical. In a special embodiment, the catalyst bed is located in a tube reactor, preferably in the reactor tubes of tube bundle reactor. Suitable tubular reactors for carrying out catalytic gas-phase reactions generally contain a catalyst tube bundle that is traversed by the reaction gas, is filled with a catalyst bed according to the invention, and around which flows a heat transfer medium contained within a surrounding reactor jacket. The heat transfer medium is preferably a salt-bath, generally a melted mixture of various salts such as alkaline nitrates and/or nitrites.

The term “catalyst bed” refers to the part of a reactor or reactor tubes that is filled with catalyst particles. The volume of the catalyst bed thus comprises the combined volume of the catalyst pellets and the combined void volume between the catalyst particles and between the catalyst particles and the reactor walls or tubes. A catalyst bed may be further diluted with inert material particles. In that case the volume of the catalyst bed is meant to include the combined volume of inert particles, too. The catalyst bed may be formed from catalyst bodies differing in their shape and/or chemical composition. However, preferably all of the particles forming the catalyst bed are essentially identical (differing only within manufacturing tolerances). Therefore, the recator tube is essentially filled with catalyst particles having a homogeneous constitution.

In the context of the invention, the prefix Cₙ-Cₘ indicates the number of carbon atoms which a molecule or a radical (group) to which it refers may have.

In the context of the present invention, the expression C₁-C₁₀-alkyl groups represents linear and branched and optionally substituted alkyl groups.

Suitable C₈-C₁₀-alkyl groups are preferably selected from n-octyl, 2-ethylhexyl, n-nonyl, n-decyl and constitutional isomers thereof.

Suitable C₁-C₇-alkyl groups are in each case unbranched and branched, saturated, optionally substituted hydrocarbon radicals having 1 to 7 carbon atoms, wherein preference is given to C₁-C₆-alkyl groups, especially C₁-C₄-alkyl groups. C₁-C₆-alkyls are, for example, methyl, ethyl, n-propyl, isopropyl, n-butyl, isobutyl (2-methylpropyl), sec-butyl (1-methylpropyl), tert-butyl (1,1-dimethylethyl), n-pentyl, n-hexyl, n-heptyl and the constitutional isomers thereof. C₁-C₄-alkyl refers to methyl, ethyl, n-propyl, isopropyl, n-butyl, isobutyl and tert.-butyl. Preferably, the C₁-C₄-alkyl refers to methyl, ethyl, n-propyl and isopropyl, in particular to methyl and ethyl.

In the context of the invention, cycloalkyl refers to a cycloaliphatic radical preferably having 5 or 6, particularly preferably 6 carbon atoms. Examples of cycloalkyl groups are, particularly, cyclopentyl, cyclohexyl, especially cyclohexyl.

Substituted cycloalkyl groups may have one or more substituents (e.g. 1, 2, 3, 4, or 5) depending on the size of the ring. These are each preferably independently selected from C₁-C₆-alkyl. In the case of substitution, the cycloalkyl groups preferably bear one or more, for example one, two, three, four or five C₁-C₆-alkyl groups. Examples of substituted cycloalkyl groups are particularly 2- and 3-methyl-cyclopentyl, 2- and 3-ethylcyclopentyl, 2-, 3- and 4-methylcyclohexyl, 2-, 3- and 4-ethylcyclohexyl, 2-, 3- and 4-propylcyclohexyl, 2-, 3- and 4-isopropylcyclohexyl, 2-, 3- and 4-butylcyclohexyl, 2-, 3- and 4-isobutylcyclohexyl , 2-, 3- and 4-tert-butylcyclohexyl and 1,2,3,4,5-methylcyclohexyl.

The catalyst bed according to the invention is characterized by a high packing density of the catalyst in the volume of the reactor tubes. In the sense of the invention the expression “packing density” is defined as the mass of the catalyst bodies per volume unit of the catalyst bed. It can be determined by the total mass of the full-metallic silver catalyst bodies of the catalyst bed divided by the total volume of the catalyst bed.

The catalyst bed according to the invention is further characterized by a narrow particle size distribution of the employed catalyst bodies. Preferably, the full-metallic silver catalyst bodies of the catalyst bed have a mean particle size of 0.5 mm to 5 mm, preferably 1.0 mm to 4 mm. The particle size is determined as the size sieve range for example a particle size of 1 to 2 mm; is the fraction which is sieved between a 2 and 1 mm sieve.

The catalyst bed according to the invention is further characterized by an optimal void space ratio. The employed catalyst bodies form a multitude of channels through which the gaseous reaction mixture can flow. This avoids or diminishes inhomogeneities of the flow, in particular through the different reactor tubes of a tube bundle reactor, and the formation of coke, leading to a longer service life of the catalyst bed. In the sense of the invention the expression “void space ratio” is the percentage of the volume of the catalyst bed not occupied by the catalyst bodies per volume unit of the catalyst bed (void fraction in % =(catalyst bed volume−total combined catalyst body volume)/catalyst bed volume×100). The volume of the pores and channels that open at the surface of the catalyst bodies is not counted as part of the void space.

Preferably, the catalyst bed according to the invention have a void space ratio in the range of 5% to 70% based on the volume of the catalyst bed not occupied by the catalyst bodies per volume of the catalyst bed, more preferably in the range of 10% to 50%.

Catalyst Bodies:

The catalyst bed according to the invention comprises full-metallic silver catalyst bodies. The term “full-metallic silver catalyst” also comprises catalysts containing silver on a monolithic metal carrier.

In contrast to granulates or crystals, which usually have an irregularly shape, the full-metallic silver catalyst according to the invention has a regular shape. The shapes are defined below. Furthermore, in contrast to fibers, filaments and threads, which have a macroscopic expansion in two dimensions, the full-metallic silver catalyst according to the invention has a 3-D structure. The full-metallic silver catalyst according to the invention is neither knitted nor woven nor crumpled. Therefore, full-metallic silver catalysts according to the invention are not in form of granulates, crystals, fibers, filaments and threads.

In a preferred embodiment, the full-metallic silver catalyst does not comprise a carrier being different from the active metal.

Preferably, the full-metallic silver bodies have a geometric surface area in the range of 100 mm²/g to 600 mm²/g.

In a special embodiment, the catalyst bodies have an essentially homogeneous composition. This differentiates them from layered catalyst bodies, core shell catalysts, supported catalysts, etc.

Preferably, the catalyst bodies comprise at least 80 wt.-%, more preferably at least 85 wt.-%, in particular at least 89 wt.-% silver, based on the total weight of the catalyst bodies, especially at least 99 wt.-% silver, based on the total weight of the catalyst bodies. Preferably, the catalyst bodies comprise 80.0 to 100.0 wt.-%, more preferably 85.0 to 100.0 wt.-%, in particular 89.0 to 100.0 wt.-% silver, based on the total weight of the catalyst bodies. In a special embodiment, the catalyst bodies comprise 89.0 to 99.9 wt.-% silver, especially 90.0 to 93.5 wt.-% silver, very special 92.5 wt.-% silver, based on the total weight of the catalyst bodies (sterling-silver).

The catalyst bodies may be partially oxidized at the surface for example when prepared under an air atmosphere.

Besides silver, the catalyst bodies may comprise one or more promotor elements. A promotor element denotes a component that provides an improvement in one or more of the catalytic properties of the catalyst when compared to a catalyst not containing said component. The promotor elements can be any of those species known in the art that function to improve the catalytic properties of the silver catalyst. Examples of catalytic properties include operability (resistance to runaway), selectivity, activity, turnover and catalyst longevity.

Promoted catalyst bodies comprise preferably 0.01 wt.-% to 20 wt.-%, more preferably 0.1 wt.-% to 15 wt.-%, in particular 1 wt.-% to 11 wt.-%, of promoter elements based on the reduced metallic form of the promoter elements and the total weight of the catalyst bodies.

The dopant preferably comprises at least one promoter element selected from B, Al, Zn, Si, Ge, In, Ti, Ta, Zr, V, Cr, Mo, W, Mn, Re, Fe, Ru, Co, Rh, Ir, Ni, Pd, Pt, Cu, Sn, Ag, Au, Ce, Cd, Pb, Na and Bi.

Full-metallic silver bodies as described in the following specific embodiments are commercially available, e.g. from Sigma Aldrich.

In a special embodiment, the full-metallic silver bodies consist essentially of 89 to 93.5 wt.-% silver, 0.1 to 2 wt.-% silicon, 0.001 to 2 wt.-% boron, 0.5 to 5 wt.-% zinc, 0.5 to 6% wt.-copper, 0.25 to 2 wt.-% tin, and 0.01 to 1.25 wt.-% indium, based on the total weight of the full-metallic silver bodies. The percentage of silver may be varied depending upon the quality of the alloy to be produced. The above ranges encompass both coin silver (i.e., containing at least 90% silver) and sterling silver (i.e., containing at least 92.5% silver).

In one specific embodiment, the full-metallic silver bodies consist essentially of about 92.5% wt.-% silver, about 0.5 wt.-% copper, about 4.25 wt.-% zinc, about 0.02 wt.-% indium, about 0.48 wt.-% tin, about 1.25 wt.-% of a boron-copper alloy containing about 2 wt.-% boron and about 98 wt.-% copper, and about 1 wt.-% of a silicon-copper alloy containing about 10 wt.-% silicon and about 90 wt.-% copper, based on the total weight of the full-metallic silver bodies. In other words, the metallic silver body consists essentially of: about 92.5 wt.-% silver, about 2.625 wt.-% copper, about 4.25 wt.-% zinc, about 0.02 wt.-% indium, about 0.48 wt.-% tin, about 0.025 wt.-% boron, and about 0.1 wt.-% silicon, based on the total weight of the full-metallic silver bodies.

In one specific embodiment, the full-metallic silver bodies consist essentially of about 99.979 wt.-% silver, at the most 0.0030 wt.-% copper, at the most 0.0010 wt.-% iron, at the most 0.0010 wt.-% zinc, at the most 0.0020 wt.-% cadmium, at the most 0.0010 wt.-% nickel, at the most 0.0020 wt.-% palladium, at the most 0.0010 wt.-% platinum, less than 0.0010 wt.-% bismuth, less than 0.0010 wt.-% lead, at the most 0.0010 wt.-% tellurium, less than 0.0010 wt.-% indium and at the most 0.0060 wt.-% sodium, based on the total weight of the full-metallic silver bodies.

In one special embodiment, the full-metallic silver bodies consist essentially of about 99.9886 wt.-% silver, about 0.0021 wt.-% copper, about 0.0002 wt.-% iron, about 0.0002 wt.-% zinc, about 0.0010 wt.-% cadmium, about 0.0005 wt.-% nickel, about 0.0015 wt.-% palladium, about 0.0002 wt.-% platinum, less than 0.0001 wt.-% bismuth, less than 0.0002 wt.-% lead, about 0.0001 wt.-% tellurium, less than 0.0001 wt.-% indium and 0.0052 wt.-% sodium, based on the total weight of the full-metallic silver bodies.

The metallic silver bodies may be prepared by the processes described in, for example, U.S. Pat. Nos. 3,019,485, 5,154,220 and 2,758,360.

Alternatively, a silver wire is heated to at least its melting temperature at one end such that the molten silver drops off to give a rounded silver material.

The rounded silver bodies can also be prepared by cutting of silver wire, or grinding other silver sources, and deforming the cut pieces to resemble a rounded polygonal or smooth shape (US 2008/0286469).

In a preferred embodiment, the geometry of the metallic silver bodies are designed in such a way that they have rounded edges. Rounded edges, in the sense of the presently claimed invention, refer to edges which are more flowing rather than jagged or angular, which means the contour is of a closed curve or the surface has no sharp corners, such as in case of an ellipse, a circle, a rounded rectangle or a sphere.

Preferably, the geometric shape of the metallic silver bodies is selected from a cylindrical shape, spherical shape, sphere-like shape or combinations thereof. It goes without saying that in reality they do not have an ideal geometric form, but rather approach the ideal shape.

A cylindrical shape in the sense of the presently claimed invention refers to a shape relating to or having the form or properties of a cylinder.

A spherical shape in the sense of the presently claimed invention means a rounded but not perfectly round shape in the three-dimensional space.

A sphere-like shape in the sense of the presently claimed invention has continuous surface, i.g. for every point of the surface a tagent can be defined. Examples of a sphere-like shape is a droplet shape or ovoid shape.

A droplet shape in the sense of the presently claimed invention means a more or less spherical or pear-like shape.

In a special embodiment, the geometric shape of the metallic silver bodies approach the ideal shape of a sphere. A person skilled in the art generally refers to this material as silver “shot” or silver “casting grain”.

These silver spheres (slover shots) have a droplet-like appearance and have a size distribution corresponding to a sieve fraction preferably in the range of from 0.1 cm to 0.4 cm, preferably in the range of 0.2 cm to 0.3 cm. The synthesis of such material is possible by melting metallic silver and then pouring through a sieve. The sieved molten silver then adopts a spherical—droplet like—morphology. The molten silver droplets are then solidified by cooling through a cooling medium, like water, near the sieve. Various cooling mediums and gas atmospheres can be used. The use of air as atmosphere is preferred.

Catalyst Bed:

According to the invention a catalyst bed, comprises full-metallic silver bodies, wherein the catalyst bed has a packing density of the full-metallic silver bodies in the range of 3.0 g/cm³ to 10.0 g/cm³, preferably 4.0 g/cm³ to 9.0 g/cm³.

In a preferred embodiment, the catalyst bed has a packing density of the metallic silver bodies preferably in the range of 4.5 g/cm³ to 9.0 g/cm³, more preferably in the range of 4.5 g/cm³ to 8.5 g/cm³, even more preferably in the range of 5.0 g/cm³ to 8.5 g/cm³, most preferably in the range of 5.5 g/cm³ to 8.5 g/cm³, particularly in the range of 5.5 g/cm³ to 8.0 g/cm³, especially in the range of 5.5 g/cm³ to 7.0 g/cm³.

In a special embodiment the catalyst bed as defined above and below has a packing density of the full-metallic silver catalyst bodies in the range of 5.5 g/cm³ to 10.0 g/cm³, preferably 6.0 g/cm³ to 10.0 g/cm³.

One process for determining the packing density is specified in the method section which follows. It has thus been found that the use of inventive catalyst bed having a packing density as defined above allows to achieve a particularly high reactor performance. It is assumed, without a restriction of the invention thereto, that the claimed packing density corresponds to a particularly favourable packing structure of the metallic silver bodies. The compact structure of the catalyst bed and the high packing density as well as the relatively low surface area, have beneficial impact on the thermal profile of the catalyst bed and they limit the residence time of the thermally unstable products in the catalyst bed. In a particularly preferred inventive embodiment, the inventive catalyst bed used will thus, in addition to the geometric surface area described herein and the size distribution of the metal silver body, also have the packing density defined herein.

In a preferred embodiment, the catalyst bed according to the invention is located in a tube reactor, preferably in the reactor tubes of a tube bundle reactor.

Tubular Reactor

Another aspect of the invention is a tubular reactor comprising a catalyst bed as defined above and below.

In a preferred embodiment, the reactor, comprising a plurality of reactor tubes, containing a catalyst bed as defined above and below.

The process is preferentially performed in a reactor as described in EP 0 881 206 B1, consisting of many short tubular reactors which are placed in a salt bath. As the reagents and products of the process are thermally unstable, it is preferred to have relatively short reactor tubes to minimize residence times. It is also preferred to have relatively thin reactor tubes to maximize cooling through the salt bath and thus to minimize the hotspot temperature linked to the highly exothermic nature of the reaction. If the process was performed without cooling through a salt bath, the high temperatures obtained under adiabatic conditions would be detrimental to the selectivity.

The inventive catalyst bed is simple to introduce into the reactor especially in the case of spheres. A further advantage of the regular shape of the catalyst is that, without further measures, orderly close packing is obtained in the reactor and, in the case of tube bundle reactors, each individual tube of the bundle exhibits a very similar pressure drop owing to the uniformity of packing. The identical pressure drop arising in many tubes of a tube bundle reactor leads to equal flow through the individual pipes and thereby evidently to a significant improvement in the selectivity of the reaction. Individual pipes do not experience higher space velocities, so that the on-stream time of the catalyst under the conditions of the invention is very high, a number of years in practice.

The term “plurality of tubes”, in the sense of the presently claimed invention means the number of tubes (or pipes) in a tubular reactor. Such tubes might be round, ellipsoid of angular, preferably round or ellipsoid.

Depending on the desired reactor capacity, the tube bundle reactor used has typically from 5 to 60000 tubes, preferably from 500 to 50000 tubes, in particular from 1000 to 45000 tubes. For experimental purposes, a single tube can be used.

In yet another embodiment, the tubular reactor comprising a plurality of tubes arranged between tube sheets.

The term “tube sheet” means the round flat piece of a plate or a sheet with holes drilled to accept the tubes or the pipes in an accurate location and a pattern relative to one another. The “tube sheets” are used to support and isolate tubes in the tubular reactor.

In a preferred embodiment, the reaction tubes have an inside diameter preferably in the range from 0.25 cm to 5.0 cm; more preferably in the range from 0.5 cm to 4.0 cm; particularly in the range of from 1.0 cm to 1.5 cm.

Preferably, the reaction tubes have a length in the range of at least 5, preferably from 10 to 60, cm, especially from 20 to 40 cm, in length.

Preferably, the packing height of the inventive catalyst bed in the reaction tube in the range of 12 mm to 500 mm, especially, 50 mm to 500 mm. However, the packing height depends on the length and the inside diameter of the reaction tubes. The height of the catalyst bed is preferably somewhat shorter than the length of the tubes. Preferably it extends only in parts of the tube which are well heat-exchanged with the external cooling-medium. In a preferred embodiment part of the reactor tube towards the inlet and/or outlet of individual tubes may be filled with shaped bodies of a more or less inert material such as steatite. In a preferred embodiment these inert materials have shapes similar to the catalyst particles. Preferably all reactor tubes are filled in a way as similar to each other in terms of pressure drop, catalyst bed volume and position of the catalyst bed and, if present, inert material. Filling of individual tubes can be simplified using pre-bagged samples of the catalyst with a defined volume of catalyst or inert materials per bag. Pressure drop of individual tubes may be monitored and documented for quality control. A limited number of tubes may be filled in a modified way in order to accommodate to thermo conditions.

The heat transport properties in reaction tubes which are filled with a solid packing is calculated in the prior art based on quasi homogeneous models of the catalyst packing. A person skilled in the art applies the so called Λr(r)-α_{w}-Model (VDI-GVC (ed.), VDI-Heat Atlas, Chapter M7, Springer-Verlag, 2010). This model considers the dependency of the radial thermal conductivity Λᵣ of the packing and heat transfer α_{w} at the tube wall on the fluid flow, the physical properties of the fluid phase, the material properties and the structural properties of the solid packing.

In a preferred embodiment, the inventive catalyst beds in the reactor tubes have a heat conductivity (radial thermal conductivity) in the range of 1.0 to 1.5 W/mK.

In a preferred embodiment, the inventive catalyst beds in the reactor tubes have a heat transfer value a_{w} in the range of 1000 to 1550 W/m²/K.

The residence time of the gas mixture in the reaction tube is preferably within the range from 0.0005 to 2, more preferably within the range from 0.001 to 1.5 seconds. The composition of the reaction gas is described in detail below.

Preferably, concentration of combustible molecules, oxygen and inert gases are defined by controlling feed composition in a way to avoid entering an explosive regime in the process. Preferably, this is realized by running “fat” composition with a concentration of combustible molecules above the explosive limit. To avoid running in the explosive regime upon start-up, nitrogen can be used instead of air. Once steady state operation is obtained, nitrogen can slowly be exchanged by air.

Process:

Another aspect of the invention is a process for the preparation of an olefinically unsaturated carbonyl compound in a tubular reactor comprising a plurality of tubes, comprising at least the following process step of treating an olefinically unsaturated alcohol with oxygen or an oxygen containing gas mixture, preferably air, in the presence of a catalyst bed as defined above.

In a preferred embodiment the process according to the invention relates to the process for the preparation of olefinically unsaturated carbonyl compounds, wherein the carbonyl compound is an α,β- and/or β,γ-olefinically unsaturated aldehyde and the olefinically unsaturated alcohol is an α,β- and/or β,γ-olefinically unsaturated alcohol.

Generally, the starting materials used in the process are commercially available or can be prepared by methods known in the literature or by a skilled person.

Suitable starting compounds are compounds of formula (II.a), (II.b) and mixtures thereof.

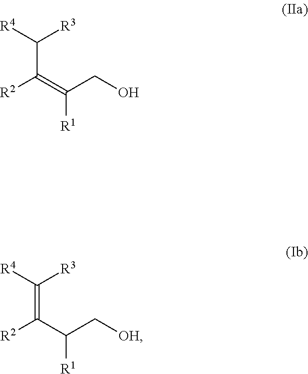

wherein

R¹, R², R³ and R⁴ are, identical or different, selected from the group consisting of H; substituted or unsubstituted C₁-C₁₀-alkyl and substituted or unsubstituted C₃₋₁₀-cycloalkyl;

or

R¹ and R² together with the carbon atoms to which they are bonded form a substituted or unsubstituted, 5- or 6-membered carbocyclic ring;

or

R² and R⁴ together with the carbon atoms to which they are bonded form a substituted or unsubstituted, 5- or 6-membered carbocyclic ring;

or

R⁴ and R³ together with the carbon atoms to which they are bonded form a substituted or unsubstituted, 5- or 6-membered carbocyclic ring.

Preferably, R¹ is selected from H and C₁₋₄-alkyl, preferably H;

R² is selected from H and C₁₋₄-alkyl, preferably C₁₋₂-alkyl, especially CH₃;

R³ is selected from H and C₁₋₄-alkyl, preferably H;

R⁴ is selected from H and C₁₋₄-alkyl, preferably H.

In a specially embodiment, R¹ is H; R² is CH₃; R³ is H and R⁴ is H.

The alcohol compounds are known compounds and obtainable by known methods.

The oxidative dehydrogenation of the olefinically unsaturated alcohol as defined above leads to an olefinically unsaturated carbonyl compound, preferably to an α,β- and/or β,γ-olefinically unsaturated aldehyde.

In a preferred embodiment the olefinically unsaturated carbonyl compound is selected from a compound of formula (Ia), formula (Ib) and mixtures thereof

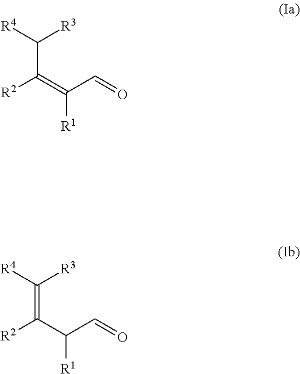

wherein

R¹, R², R³ and R⁴ are, identical or different, selected from the group consisting of H;

substituted or unsubstituted C₁-C₁₀-alkyl and substituted or unsubstituted C₃₋₁₀-cycloalkyl;

or R¹ and R² together with the carbon atoms to which they are bonded form a substituted or unsubstituted, 5- or 6-membered carbocyclic ring;

or R² and R⁴ together with the carbon atoms to which they are bonded form a substituted or unsubstituted, 5- or 6-membered carbocyclic ring;

or

R⁴ and R³ together with the carbon atoms to which they are bonded form a substituted or unsubstituted, 5- or 6-membered carbocyclic ring.

Preferably, R¹ is selected from H and C₁₋₄-alkyl, preferably H;

R² is selected from H and C₁₋₄-alkyl, preferably C₁₋₂-alkyl, especially CH₃,

R³ is selected from H and C₁₋₄-alkyl, preferably H,

R⁴ is selected from H and C₁₋₄-alkyl, preferably H.

In a specially embodiment, R¹ is H; R² is CH₃; R³ is H and R⁴ is H.

Processes and reactors suitable for oxidation of unsaturated alcohols are known to a person skilled in the art. The afore-mentioned catalyst beds and reactors are generally suitable for the known precesses, as described in e.g. EP 0 881 206.

Generally the process for the preparation of an olefinically unsaturated carbonyl compounds comprises the steps:
a) vaporizing of the olefinically unsaturated alcohol, preferably compound (IIa), (IIb) or mixtures thereof, in particular prenol and/or isoprenol;
b) admixing the alcohol vapor, provided in step a) with an oxygen-comprising gas;
c) passing the resulting gas, comprising oxygen and the vapor of the alcohol component, through a layer of the inventive catalyst bed as defined above,
d) reacting the gas, comprising oxygen and the vapor of the alcohol component in a tube bundle reactor comprising a sufficient number, for the desired capacity, of reaction tubes which are packed with the catalyst bed to form a mixture of the corresponding olefinically unsaturated carbonyl compound, preferably compound (Ia), (Ib) or mixtures thereof, in particular prenal and/or isoprenal, and
e) optionally isomerizing the isoprenal present in the resulting mixture of prenal and isoprenal into prenal in a conventional manner.

The dehydrogenation is preferably carried out at a pressure within the range from 1 to 2 bar (absolute), preferably at atmospheric or somewhat elevated pressure in order to provide for down-stream pressure drop. Especially preferred the reactor is operated in a range from 1.150 to 1.350 bar (absolute). The pressure drop along the reactor tubes is preferably maintained in a range from 5 to 100 mbar.

The dehydrogenation is preferably carried out at a temperature in the range from 300° C. to 500° C., more preferably at a temperature in the range of 350° C. to 450° C.

The dehydrogenation is usually performed in a continuous manner.

The reaction mixture, as described above, is worked up in a conventional manner. For example, the hot reaction gases are absorbed with a solvent such as water or preferably in condensed product mixture directly on emergence from the reactor.

The process of the invention makes it possible to produce the α,β-unsaturated aldehydes, especially prenal and iso-prenal, which are sought after as intermediates for the synthesis of scents, vitamins and carotenoids in good yields in advantageously fabricable tube bundle reactors with catalyst on-stream times of several years.

Generally, a regeneration cycle is performed periodically, to remove accumulated coke. The regeneration cycle can be initiated when an increase in pressure drop is noticed, or at arbitrary time intervals, for example once a week. A regeneration cycle consists of sending diluted air or air for a defined period of time, for example 6 to 24 h,over the reactor while increasing the salt bath temperature, for example 400 to 450° C., to allow coke combustion.

Another aspect of the invention is the use of a catalyst bed as defined above for the preparation of olefinically unsaturated carbonyl compounds from olefinically unsaturated alcohols by oxidative dehydrogenation.

Preferably, the catalyst bed according to the invention is used for the preparation of 3-methyl-3-buten-1-al (isoprenal) or 3-methylbut-2-enal (prenal), from 3-methyl-3-butene-1-ol (isoprenol) or 3-methylbut-2-en-1-ol (prenol) by oxidative dehydrogenation.

The present invention is now illustrated in further detail by the following examples, without imposing any limitation thereto.

### EXAMPLES

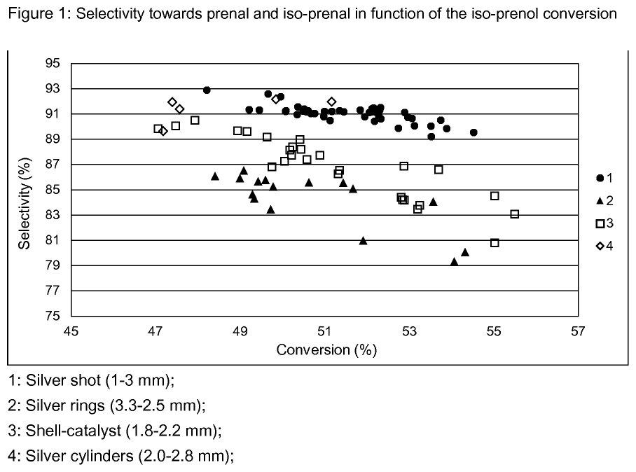

FIG. 1: Selectivity towards prenal and iso-prenal as a function of the iso-prenol conversion, for the catalysts described in the examples

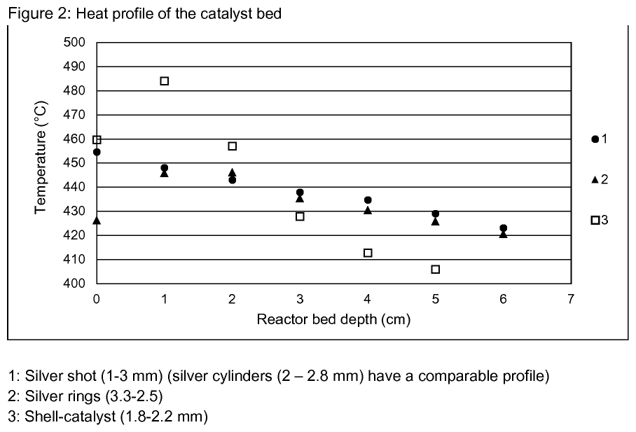

FIG. 2: Heat profile of the catalyst bed of examples under operation

Analytics:

A) Method for determining the packing density:

A glass tube with an inner diameter of 13 mm is filled with a material of interest to a defined packing height. The mass of the packed material is divided by the inner volume of the tube corresponding to that packing height.

B) Method for determining the geometric surface area ranges in the case of sphere-like catalyst bodies:

The geometric surface area ranges of catalyst bodies are calculated by assuming ideal sphericity of the catalyst bodies and using minimum and maximum diameters of a corresponding sieve fraction. The specific density of silver is used to calculate mas-specific geometric surface areas expressed in mm²/g.

C) Method for determining the size distribution (sieve fraction):

The size distribution, expressed as sieve fraction, is measured using sieves with defined sieve sizes. For example: a material with a sieve fraction of 1 to 4 mm will pass through a sieve with a sieve size ≥4 mm and will be completely retained by a sieve with a sieve size of ≤1 mm.

D) Method for determining void fraction in the tubular reactor:

The void fraction is calculated starting from the density of the catalyst bed. The combined volume of catalyst particles in the catalyst bed is calculated using the intrinsic material density (specific density) of the material. In the case of silver we have used a value of 10.5 g/ml. The void fraction is then the ratio between the void volume (volume of the catalyst bed minus the calculated combined volume of all the catalyst bodies in the catalyst bed) and the volume of the catalyst bed.

E) Method for determining the weight of the catalyst bed

A glass tube with an inner diameter of 13 mm is filled with a material of interest to a defined packing height. The mass of the packed material is then measured.

Oxidative Dehydrogenation

A setup was used comprising a continuous alcohol evaporation chamber where the educt was evaporated and mixed with air, after which the gaseous reagent was directed to a quartz reactor. The reactor had an internal diameter of 13 mm and held the catalyst bed by a metal sieve. The reactor contained a central thermocouple placed inside a glass tube (OD 3 mm), which went through the length of the catalyst bed. The catalyst bed length was kept at 7 cm. The reactor was surrounded by a chamber which was heated by an electric heating coil. This chamber contained sand which could be fluidized by a nitrogen flow, which was used to control the temperature of the reactor. Initially, the reactor was heated by the sand bath to ignite the reaction. Once the reaction was started, the fluidized sand bath was used as cooling medium to remove heat from the reactor, originating from the highly exothermic oxidation of the alcohol. A water-cooled condensation chamber was placed immediately after the reactor where the unconverted reagent and condensable products were accumulated. This condensate was periodically analyzed by a gas chromatographer. The non-condensable products left the condensation chamber and are monitored with an on-line gas chromatographer.

### Example 1 (according to the invention)

Fully metallic silver shot (1-3 mm, Sigma-Aldrich, ≥99.99%) was placed inside the above described reactor to obtain a catalyst bed length of 7 cm. 110 g/h of isoprenol was evaporated and mixed with 50 NL/h of air. This reagent stream was sent to the reactor which was heated at 360° C. After 3 hours of operation, the sandbath temperature was adjusted between 380 and 400° C. to obtain iso-prenol conversion levels between 45 and 60%. At an iso-prenol conversion of 50%, a prenal and iso-prenal selectivity of 91% was obtained. The results are depicted in table 1 below.

### Example 2 (according to the invention)

Fully metallic silver cylinders (height=2.8 mm, diameter=2 mm, Sigma-Aldrich, 9.99%) were placed inside the above described reactor to obtain a catalyst bed length of 7 cm. The material was initially ordered as a longer rod which was cut to the defined length. 110 g/h of isoprenol was evaporated and mixed with 50 NL/h of air. This reagent stream was sent to the reactor which is heated at 360° C. After 3 hours of operation, the sandbath temperature was adjusted between 380 and 400° C. to obtain iso-prenol conversion levels between 45 and 60%. At an iso-prenol conversion of 50%, a prenal and iso-prenal selectivity of 92% was obtained. The results are depicted in table 1 below.

### Example 3 (not inventive)

A “shell-catalyst”, as described in EP 263385 B1, comprising 5 wt.-% of silver coated on a spherical steatite carrier (1.8-2.2 mm), was placed inside the above described reactor to obtain a catalyst bed length of 7 cm. 110 g/h of iso-prenol was evaporated and mixed with 50 NL/h of air. This reagent stream was sent to the reactor which is heated at 360° C. After 3 hours of operation, the sandbath temperature was adjusted between 380 and 400° C. to obtain iso-prenol conversion levels between 45 and 60%. At an iso-prenol conversion of 50%, a prenal and iso-prenal selectivity of 87.5% was obtained. The results are depicted in table 1 below.

### Example 4 (not inventive)

Fully metallic silver rings (height =3 mm, outer diameter =3 mm, inner diameter=2.5 mm, Sigma-Aldrich, ≥99.99%) were placed inside the reactor described above to obtain a catalyst bed length of 7 cm. The material was initially ordered as a longer tube which was cut to the defined length. 110 g/h of isoprenol was evaporated and mixed with 50 NL/h of air. This reagent stream was send to the reactor which was heated at 360° C. After 3 hours of operation, the sandbath temperature was adjusted between 380 and 400° C. to obtain iso-prenol conversion levels between 45 and 60%. At an iso-prenol conversion of 50%, a prenal and iso-prenal selectivity of 85% was obtained. The results are depicted in table 1 below.

### Example 5 (not inventive)

Fully metallic silver crystals as described in EP 0 244 632 in two different sieving fractions (0-1 mm and 1-2 mm). Such silver crystals have a rather undefined, needle-like, appearance. This material leads to low packing densities (void fraction above 80%) and a broad spread in pressure drop over different tubes. The practical application of this material as catalyst is therefore not desired.

TABLE 1
Packing density and void fraction of selected materials

| | | | Packing | |
|---|---|---|---|---|
| | | Size range | density | Void fraction |
| Catalyst | Example | (mm) | (g/mL) | (%) |
| Shell catalyst¹ | 3 | 1.8-2.2 | 1.4 | 40.8 |
| Silver crystals | 5 | 0.2-1.0 | 2.1 | 80.1 |
| Silver crystals | 5 | 1.0-2.0 | 2.1 | 80.1 |
| Silver rings¹ | 4 | 3.0; 3.0; 2.5² | 1.4 | 86.5 |
| Silver cylinders¹ | 2 | 2.0; 2.8³ | 6.1 | 41.9 |
| Silver shot¹ | 1 | 1.0-3.2 | 6.0 | 46.2 |
| Silver shot | 1 | 1.5-2.5 | 6.3 | 40.3 |

¹Performance shown in performance examples
²Outer diameter; height; inner diameter
³Diameter; height

Discussion

Table 1 lists five different materials of which two have two different sieve size ranges. The ‘shell catalyst’ consists of supported silver on steatite spheres, as in EP 263385 B1. The ‘silver crystals’ are fully metallic particles, as in EP 244632 B1. A person skilled in the art generally refers to this material as electrolytic silver or cathode silver. This material leads to low packing density (<4 g/mL) and has the disadvantage to lead to unsatisfactory pressure drop differences between the individual tubes of a multitubular reactor. The silver rings of example 4 are fully metallic bodies which lead to a low packing density (g/mL). The performance examples demonstrate that, using these silver rings as catalyst, no improvement in selectivity is observed in comparison to the prior art. Silver cylinders and silver shot (mainly round silver bodies) are fully metallic bodies which lead to high packing densities (≥4 g/mL). The performance examples demonstrate that, using silver shot or silver cylinders, a significant improvement in selectivity is observed in comparison to the prior art.

The table 2 lists the value of the parameters Λᵣ, and α_{w} under typical operation conditions over a shell-type catalyst bed as described in EP 263385 and for a catalyst bed according to the invention.

TABLE 2

| | Λᵣ in (W/m/K) | α_{w} in (W/m²/K) |
|---|---|---|
| Coated - shell type - catalyst | 0.561 | 530 |
| Ag on steatite, 2 mm spheres | | |
| Fully metallic silver bodies | 1.34 | 1465 |
| 2 mm spheres according to the | | |
| invention | | |

1.-18. (canceled)

19. A process for the preparation of an olefinically unsaturated carbonyl compound in a tubular reactor comprising a plurality of reactor tubes, comprising reacting an olefinically unsaturated alcohol with oxygen in the presence of a catalyst bed, comprising full-metallic silver catalyst bodies, wherein the catalyst bed has a packing density of the full-metallic silver catalyst bodies in the range of 3.0 g/cm³ to 10.0 g/cm³.

20. The process according to claim 19, wherein the catalyst bed has a packing density of the full-metallic silver catalyst bodies in the range of 5.5 g/cm³ to 10.0 g/cm³.

21. The process according to claim 19, wherein the catalyst bed has a void space ratio in the range of 5% to 70%, based on the volume of the catalyst bed not occupied by the catalyst bodies per volume of the catalyst bed.

22. The process according to claim 19, wherein the full-metallic silver catalyst bodies have a mean particle size of 0.5 mm to 5.0 mm.

23. The process according to claim 19, wherein the full-metallic silver bodies have a cylindrical shape or spherical shape or sphere-like shape or combinations thereof.

24. The process according to claim 19, wherein the full-metallic silver bodies have a geometric surface area in the range of 100 mm²/g to 600 mm²/g.

25. The process according to claim 19, wherein the catalyst bed is located in a tube reactor.

26. The process according to claim 19, wherein the olefinically unsaturated carbonyl compound is an α,β- and/or β,γ-olefinically unsaturated aldehyde and the olefinically unsaturated alcohol is an α,β- and/or β,γ-olefinically unsaturated alcohol.

27. The process according to claim 19, wherein the unsaturated carbonyl compound is an olefinically unsaturated aldehyde, selected from a compound of formula (Ia), formula (Ib) and mixtures thereof 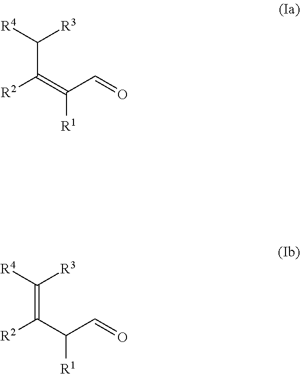
wherein
R¹, R², R³ and R⁴ are, identical or different, selected from the group consisting of H, substituted or unsubstituted C₁-C₁₀-alkyl and substituted or unsubstituted C₃₋₁₀-cycloalkyl;
or R¹ and R² together with the carbon atoms to which they are bonded form a substituted or unsubstituted, 5- or 6-membered carbocyclic ring;
or R² and R⁴ together with the carbon atoms to which they are bonded form a substituted or unsubstituted, 5- or 6-membered carbocyclic ring;
or R⁴ and R³ together with the carbon atoms to which they are bonded form a substituted or unsubstituted, 5- or 6-membered carbocyclic ring.

28. The process according to claim 27, wherein
R¹ is selected from H and C₁₋₄-alkyl,
R² is selected from H and C₁₋₄-alkyl,
R³ is selected from H and C₁₋₄-alkyl,
R⁴ is selected from H and C₁₋₄-alkyl.

29. A catalyst bed as defined in claim 19, wherein the catalyst bed has a packing density of the full-metallic silver catalyst bodies in the range of 5.5 g/cm³ to 10.0 g/cm³.

30. A catalyst bed according to claim 29, wherein the full-metallic silver bodies have a geometric surface area in the range of 100 mm²/g to 600 mm²/g.

31. The catalyst bed according to claim 29, wherein the catalyst bed is located in a tube reactor.

32. A reactor, comprising a plurality of reactor tubes containing a catalyst bed as defined in claim 29.

33. The reactor according to claim 32, wherein the catalyst beds have a radial thermal conductivity Ar in the range of 1.0 to 1.5 W/m/K.

34. The reactor according to claim 32, wherein the catalyst beds have a heat transfer value α_{w} in the range of 1000 to 1550 W/m²/K.

35. A method for preparing olefinically unsaturated carbonyl compounds from olefinically unsaturated alcohols comprising oxidatively dehydrogenating over the catalyst bed according to claim 29.

36. The method according to claim 35 where the olefinically unsaturated carbonyl compounds are 3-methyl-3-buten-1-al (isoprenal) or 3-methylbut-2-enal (prenal), and where the olefinically unsaturated alcohols are 3 -methyl-3 -butene-1-ol (isoprenol) or 3 -methylbut-2-en-1-ol (prenol).

37. The process according to claim 19, wherein the catalyst bed has a void space ratio in the range of 10% to 50%, based on the volume of the catalyst bed not occupied by the catalyst bodies per volume of the catalyst bed.

38. The process according to claim 19, wherein the full-metallic silver catalyst bodies have a mean particle size of 1.0 mm to 4.0 mm.
